(19) United States
(12) Patent Application Publication
(10) Pub. No.: US 2012/0143292 A1
(43) Pub. Date: Jun. 7, 2012

(54) TREATMENT DEVICE

(75) Inventor: Atsushi Matsuo, Osaka-shi (JP)

(73) Assignee: KOBAYASHI PHARMACEUTICAL CO., LTD., Osaka-shi (JP)

(21) Appl. No.: 13/389,511

(22) PCT Filed: Sep. 9, 2010
(86) PCT No.: PCT/JP2010/065503

§ 371 Date: Feb. 8, 2012

(30) Foreign Application Priority Data
Sep. 9, 2009 (JP) 2009-208504

(51) Int. Cl. A61F 7/00

(52) U.S. Cl. 607/96

(57) ABSTRACT

This invention relates to a treatment device that can be prevented from being displaced from a treatment site. The treatment device accommodates one or more heating elements that generate heat, and is attachable to a treatment site of a patient, the device comprising a body part formed of an inelastic and breathable material, and having one or more storage portions individually storing each heating element; one or more band parts formed of an elastic and breathable material, and attached to the body part; and a fastening means for fastening the one or more band parts wound around the treatment site.

### TECHNICAL FIELD

The present invention relates to a treatment device accommodating a heating element that generates heat, and attachable to a treatment site of a patient.

### BACKGROUND ART

Heating elements using heat generated by oxidation of metal (e.g., iron), for example, so-called disposable body warmers, provide an easy way to get warm. However, conventional heating elements attached to clothes with an adhesive may detach from the clothes as adhesion decreases. In addition, they are difficult to adhere to joints, such as the knee and elbow.

To overcome these drawbacks, PTL 1 discloses a therapeutic body wrap obtained by forming an elastic material into a band. The center of the wrap is provided with pockets for storing heating elements. The wrap is used in such a manner that heating elements are placed in the pockets, and both ends of the wrap are then wound around a treatment site.

### CITATION LIST

### Patent Literature

PTL 1: U.S. Patent Publication No. 2007/0032848

### SUMMARY OF INVENTION

### Technical Problem

However, because it is formed of an elastic material, the above wrap may follow the movement of the body, and consequently be displaced from the treatment site. The present invention was made to solve this problem, and an object of the present invention is to provide a treatment device that can be prevented from being displaced from the treatment site.

### Solution to Problem

The present invention relates to a treatment device accommodating one or more heating elements that generate heat, and attachable to a treatment site of a patient, the device comprising a body part formed of an inelastic and breathable material and having one or more storage portions individually storing each heating element, one or more band parts formed of an elastic and breathable material and attached to the body part, and a fastening means for fastening the one or more band parts wound around the treatment site.

According to this structure, the body part having a storage portion for storing a heating element is formed of an inelastic material. Therefore, the inelastic storage portion is fixed in a treatment site, such as the waist, elbow, or knee, limiting the movement of the treatment site. Accordingly, forcible movement of the treatment site is prevented, while the treatment site is warmed with the heating element. As a result, the treatment device facilitates recovery from pain and serves to prevent pain. The term “inelastic” as referred to herein does not necessarily mean that the material is completely non-extensible by application of any force, but that the material may have elasticity lower than that of the extensible band part. The term “breathable” as used for the body part and band part means that the material allows air to pass through. The material may also have moisture permeability.

As described above, to fix the movement of the treatment site, the body part preferably has one storage portion, or two to four adjacent storage portions, and more preferably one storage portion or two adjacent storage portions, per treatment site. Such a small number of storage portions can restrict the movement of the body part in the treatment site. That is, when a large number of storage portions are formed, some storage portions may move according to the movement of the treatment site; however, when the number of storage portions is smaller, as described above, the movement of the treatment site can be further limited, which is also attributable to little or no elasticity of the body part. Consequently, recovery from pain is facilitated.

Moreover, the storage portion(s) can be arranged in a rectangular configuration in the body part, per treatment site. Because of this structure, the treatment site can be easily fixed.

Furthermore, each storage portion preferably has an area of 60 cm² or more. When the area of each storage portion is larger, the movement of the treatment site can be further limited, which is also attributable to little or no elasticity of the body part. Consequently, recovery from pain is facilitated.

The treatment device of the present invention can take various forms depending on the treatment site. For example, a body part provided with a pair of band parts in the opposite end of the body part can be fixed in a relatively large treatment site. The body part can be fixed, for example, by bringing the body part into contact with the waist, and winding the pair of band parts around the trunk.

Further, a body part provided with a pair of parallel band parts in one end of the body part at a distance from each other is suitable for attachment to joints, such as the knee and wrist. More specifically, the body part can be fixed in a joint by bringing the body part into contact with the joint, and winding the band parts respectively around the limbs across the joint. The term “parallel” as referred to herein does not necessarily mean that the band parts are arranged strictly in parallel, but that they may be slightly angled as long as they are directed to the same direction as a whole.

### Advantageous Effect of Invention

The treatment device of the present invention can be prevented from being displaced from a treatment site.

### BRIEF DESCRIPTION OF DRAWINGS

FIG. 1 is a plan view of a first embodiment of the treatment device according to the present invention.

FIG. 2 is a plan view of another example of the treatment device of FIG. 1.

FIG. 3 is a plan view of a second embodiment of the treatment device according to the present invention.

FIG. 4 is a plan view of another example of the treatment device of FIG. 3.

FIG. 5 is a plan view of a third embodiment of the treatment device according to the present invention.

FIG. 6 is a plan view of another example of the treatment device of FIG. 5.

### DESCRIPTION OF EMBODIMENTS

### First Embodiment

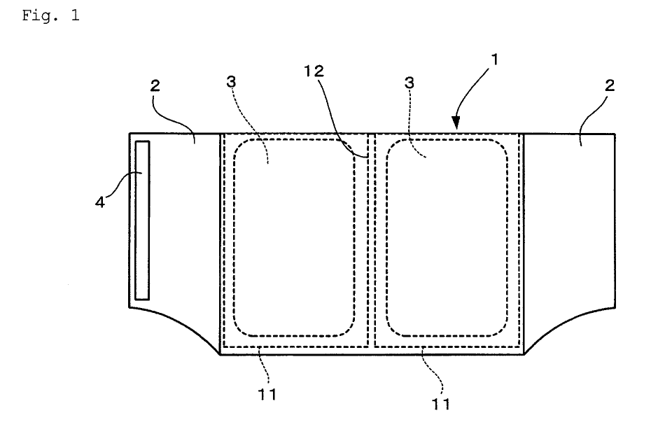

A treatment device according to the first embodiment of the present invention is described with reference to the drawings. FIG. 1 is a plan view of the treatment device of this embodiment.

The treatment device of this embodiment is mainly designed for attachment to the waist. As shown in FIG. 1, the treatment device comprises a body part 1 having two rectangular storage portions 11, and a pair of band parts 2 extending from both ends of the body part 1.

Although the specific structure of the body part 1 is not limited, the body part 1 is formed of a breathable sheet material, such as a nonwoven fabric of natural fiber or synthetic fiber. Examples of usable nonwoven fabrics include nonwoven fabrics containing polypropylene, nylon, vinylon, polyester, polyethylene terephthalate, rayon, acetate, acrylic, polyethylene, polyvinyl chloride, and other synthetic fibers; and nonwoven fabrics containing cotton, hemp, silk, and other natural fibers. Moreover, the body part 1 is inelastic. However, the body part 1 is not necessarily strictly inelastic; the material of the body part 1 may have elasticity lower than that of elastic materials, such as rubber. For example, a material (100 mm×50 mm) is prepared and pulled in the longitudinal direction. When the force required to elongate the material 150% is 45 N or more, such a material can be used. Two inelastic sheet materials are laminated to form a storage portion 11 for storing a heating element 3 (e.g., a chemical body warmer). As such a material, for example, a non-woven fabric with a weight of 30 to 100 g/m² can be used.

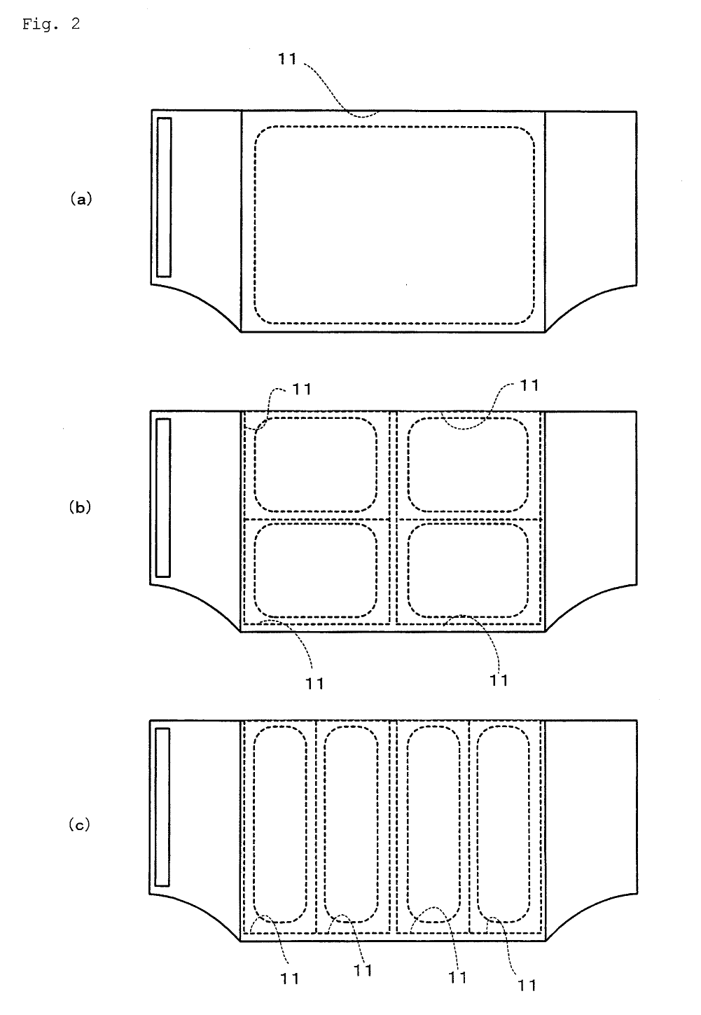

The number of storage portions 11 may be plural, and is preferably 1 to 4. When a plurality of storage portions 11 is formed, two sheet materials may be welded, for example, by ultrasonic welding 12. Although a plurality of storage portions 11 can be formed as shown, for example, in FIG. 2, the shape of each storage portion is preferably rectangular in conformity to the shape of a general heating element. Moreover, when a plurality of storage portions 11 is formed, the entire shape is preferably rectangular. Furthermore, the body part 1 may be formed of three or more sheet materials, or with one sheet material that has pockets as storage portions.

Next, the band part 2 is described. The band parts 2 are elastic sheet materials attached to both the right and left ends of the body part 1. The band parts 2 can be fixed in the body part 1, for example, by ultrasonic welding. The end portion of one band part 2 is provided with a surface fastener 4 as a fastening means. Since the target treatment site in this embodiment is the waist, the band parts 2 may be so elastic as to be wound around the trunk. For example, a material (100 mm×50 mm) is prepared and pulled in the longitudinal direction. When the force required to elongate the material 150% is about 1.5 to 70 N, such a material can be used as a sheet material. In addition, the band parts 2 are wound around the trunk, and are thus required to be air-permeable. For this purpose, a breathable material is used, or a plurality of through-holes is formed to achieve breathable performance.

Although the specific structure of the material forming the band part is not limited, a nonwoven fabric of natural fiber or synthetic fiber is preferably used. The band part may comprise not only a single layer, but several layers.

Next, the usage of the treatment device is described. First, a heating element 3 is accommodated in each storage portion 11 of the body part 1. Subsequently, the device is arranged so that the body part 1 is in contact with the waist, and both band parts 2 are stretched and wound around the trunk. Then, the stretched band parts 2 are overlaid in front of the trunk, and the surface fastener 4 of one band part 2 is fixed in the other band part 2.

As described above, according to this embodiment, the body part 1 having the storage portions 11 for storing the heating elements 3 is formed of an inelastic material; therefore, the inelastic storage portions 11 are fixed on the treatment site, i.e., waist, to restrict the movement of the waist. As a result, forcible movement of the waist is prevented, while the treatment site can be warmed with the heating elements 3. Consequently, the treatment device facilitates recovery from pain, and serves to prevent pain.

### Second Embodiment

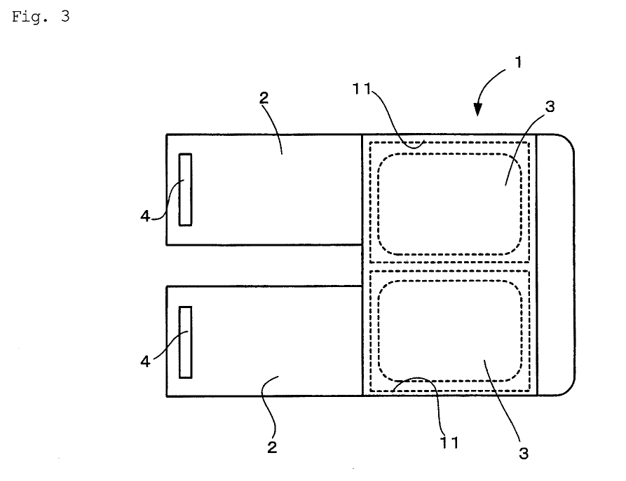

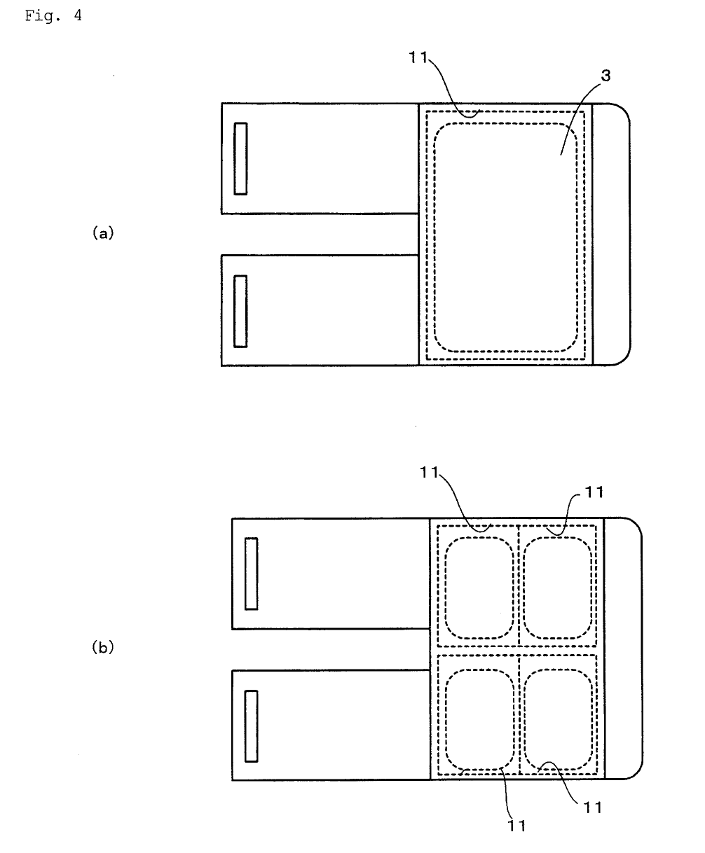

A treatment device according to the second embodiment of the present invention is described with reference to the drawings. FIG. 3 is a plan view of the treatment device of this embodiment. The treatment device of this embodiment is mainly designed for attachment to the wrist. As shown in FIG. 3, this treatment device comprises a body part 1 having two rectangular storage portions 11, and a pair of band parts 2 extending from both ends of one side of the body part 1. The body part 1 has the same structure as that of the first embodiment, that is, the storage portions 11 are formed to store heating elements 3. The number and area of storage portions 11 may be the same as those of the first embodiment, and examples are shown in FIG. 4. Both band parts 2 extend in parallel from one end of the body part 1, and the end portion of each band part 2 is provided with a surface fastener 4. The materials forming the body part 1 and band parts 2 are the same as those used in the first embodiment; therefore, the explanation thereof is omitted.

This treatment device is used in the following manner. First, a heating element is stored in each storage portion 11, and the body part 1 is brought into contact with the wrist. Here, the space between the two band parts 2 is positioned on the wrist. Then, one band part 2 is wound around the back of the hand, and fixed with the surface fastener 4. Subsequently, the other band part 2 is wound around the forearm, and fixed with the other surface fastener 4.

This embodiment also provides the same effect as that of the first embodiment, because the body part 1 is formed of an inelastic sheet material.

### Third Embodiment

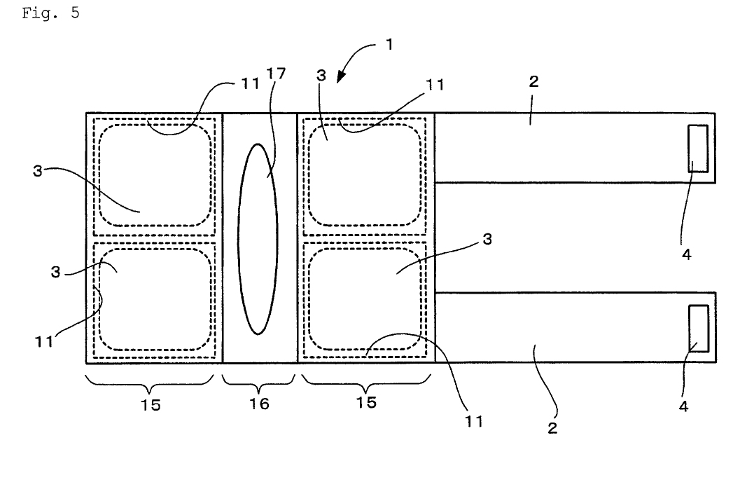

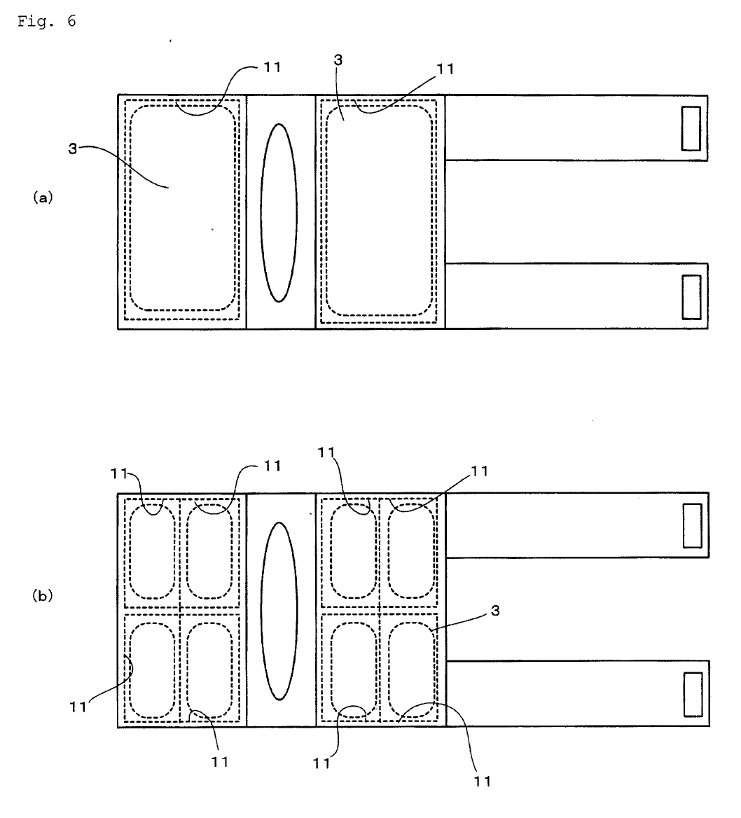

A treatment device according to the third embodiment of the present invention is described with reference to the drawings. FIG. 5 is a plan view of the treatment device of this embodiment. The treatment device of this embodiment is mainly designed for attachment to the knee. As shown in FIG. 5, this treatment device comprises a body part 1 having four rectangular storage portions 11, and a pair of band parts 2 extending from both ends of one side of the body part 1. The body part 1 is divided into three areas: storage areas 15 in which the two storage portions 11 are formed, and a knee pad area 16 that is located between the two storage areas 15 and that is to be in contact with the knee. Each of the storage area 15 has the same structure as that of the body part 1 of Embodiment 1 or 2, that is, two sheet materials are laminated to form two pockets. Other examples of the structure are shown in FIG. 6. The knee pad area 16 has an ellipsoidal through-hole 17, into which the kneecap can enter. In this embodiment, one storage area 15 is brought into contact with one treatment site.

The two band parts 2 extend in parallel from the end of one storage area 15, and the end portion of each band part 2 is provided with a surface fastener 4. The materials forming the body part 1 and band parts 2 are the same as those used in the first embodiment; therefore, the explanation thereof is omitted.

This treatment device is used in the following manner. First, a heating element 3 is stored in each storage portion 11, and the body part 1 is brought into contact with the knee. Here, the body part 1 is arranged so that the through-hole 17 of the knee pad area 16 is in contact with the kneecap. Then, one band part 2 is wound around the thigh above the kneecap and fixed with the surface fastener 4. Subsequently, the other band part 2 is wound around the area below the kneecap and fixed with the other surface fastener 4.

This embodiment also provides the same effect as that of the first embodiment, because the body part 1 is formed of an inelastic sheet material.

Although the embodiments of the present invention are described above, the present invention is not limited thereto, and various modifications can be made without deviating from the scope of the invention. For example, the surface fastener is used as the fastening means in the above embodiments; however, other means can be used without limitation, as long as they can fix the band part. Further, every band part may be provided with a surface fastener. The heating element is not necessarily a chemical body warmer, and various products, such as those driven by electricity, fuel, etc., can be used.

### EXAMPLES

### Example 1

The treatment device for the waist shown in the first embodiment was prepared. First, the materials thereof are explained.

### (1) Material

### (a) Body Part

### Polypropylene Nonwoven Fabric

Polypropylene nonwoven fabrics having a weight of 5, 10, 15, 30, 35, 50, 65, 80, or 90 g/m² were used. These fabrics were referred to as Ex. 1 to Ex. 9 in this order.

### (b) Band Part

For the band part, polypropylene spunbonded nonwoven fabrics having a weight of 60, 80, 100, 120, 130, 140, or 160 g/m² were used. These fabrics were referred to as Ex. 10 to Ex. 16 in this order.

### (2) Evaluation of Elasticity

To evaluate the elasticity, sheet materials (100 mm×50 mm) were prepared for the body part and band part. Each sheet material was pulled in the longitudinal direction using a tensile testing machine (AGS-H, produced by Shimadzu Corp.), and the force required to elongate the sheet material 150% was measured. The above-described materials were combined to produce treatment devices of the form shown in FIG. 1. Each of these treatment devices was used for one day by five subjects suffering from lumbago, and evaluated in terms of the degree of lumbago alleviation on the following three scales: very satisfactory: A, satisfactory: B, and unsatisfactory: C. The results are as follows.

TABLE 1

| Body | 115 (Ex. 9) | A | A | A | A | A | A | A |
|---|---|---|---|---|---|---|---|---|
| part | 102 (Ex. 8) | A | A | A | A | A | A | A |
| | 85 (Ex. 7) | A | A | A | A | A | A | A |
| | 63 (Ex. 6) | A | A | A | A | A | A | A |
| | 55 (Ex. 5) | A | A | A | A | A | A | A |
| | 45 (Ex. 4) | B | B | B | B | B | B | B |
| | 27 (Ex. 3) | C | C | C | C | C | C | C |
| | 5 (Ex. 2) | C | C | C | C | C | C | C |
| | 1.5 (Ex. 1) | C | C | C | C | C | C | C |
| | | 1.5 | 5 | 18 | 27 | 45 | 55 | 63 |
| | | (Ex. | (Ex. | (Ex. | (Ex. | (Ex. | (Ex. | (Ex. |
| | | 10) | 11) | 12) | 13) | 14) | 15) | 16) |

Band part
Unit: N

These results clarify that the elasticity of the body part has greater influence than the elasticity of the band part, and particularly indicate that the body part is preferably formed of a material requiring a force of 45 N or more to exhibit an elongation of 150%.

### (3) Evaluation of Storage Portion Area

Treatment devices having four types of areas, as shown in FIGS. 1 and 2, were prepared. The treatment device of Experimental Example 1 (FIG. 2(a)) had one rectangular storage portion with an area of 247 cm². The treatment device of Experimental Example 2 (FIG. 1) had two adjacent rectangular storage portions, each of which had an area of 123.5 cm². The treatment device of Experimental Example 3 (FIG. 2(b)) had four rectangular storage portions adjacent in a grid pattern, each of which had an area of 61.75 cm². The treatment device of Experimental Example 4 (FIG. 2(c)) similarly had four band-like storage portions adjacent in parallel, each of which had an area of 30.9 cm². Each of these treatment devices was used for six days by ten subjects suffering from lumbago, and evaluated in terms of the degree of lumbago alleviation on the following three scales: very satisfactory: A, satisfactory: B, and unsatisfactory: C. The results are as follows.

TABLE 2

| | Exp. Ex. 1 | Exp. Ex. 2 | Exp. Ex. 3 | Exp. Ex. 4 |
|---|---|---|---|---|
| Evaluation | A | A | B | C |

These results demonstrate the effectiveness of the treatment devices whose body parts had one or more storage portions each having an area of 60 cm² or more.

Additionally, as a result of further consideration of the results of using the treatment device of Experimental Example 2, the following results were obtained.

TABLE 3

| | | | Level of Satisfaction | | Average Number of |
|---|---|---|---|---|---|
| Degree of Lumbago | Sensation | Sense of | with Time Needed for | Total Level of | Days Needed for |
| Alleviation | of Warmth | Fitting | Pain Alleviation | Satisfaction | Pain Alleviation |
| A | A | B | A | A | 2.0 |

Furthermore, the subjects answered that their recovery seemed to be fast because the movement of the waist was fixed to thereby prevent forcible movement of the waist.

### Example 2

The treatment device for the wrist shown in the second embodiment was prepared. First, the materials thereof are explained.

### (1) Material

The same materials as those used in Example 1 were used. Thus, the explanation thereof is omitted.

### (2) Evaluation of Elasticity

To evaluate the elasticity, sheet materials (100 mm×50 mm) were prepared for the body part and band part. Each sheet material was pulled in the longitudinal direction using a tensile testing machine (AGS-H, produced by Shimadzu Corp.), and the force required to elongate the sheet material 150% was measured. The above-described materials were combined to produce treatment devices of the form shown in FIG. 3. Each of the thus-prepared treatment devices was used for one day by five subjects suffering from wrist pain, and evaluated in terms of the degree of wrist pain alleviation on the following three scales: very satisfactory: A, satisfactory: B, and unsatisfactory: C. The results are as follows.

TABLE 4

| Body | 115 (Ex. 9) | A | A | A | A | A | A | A |
|---|---|---|---|---|---|---|---|---|
| part | 102 (Ex. 8) | A | A | A | A | A | A | A |
| | 85 (Ex. 7) | A | A | A | A | A | A | A |
| | 63 (Ex. 6) | A | A | A | A | A | A | A |
| | 55 (Ex. 5) | A | A | A | A | A | A | A |
| | 45 (Ex. 4) | B | B | B | B | B | B | B |
| | 27 (Ex. 3) | C | C | C | C | C | C | C |
| | 5 (Ex. 2) | C | C | C | C | C | C | C |
| | 1.5 (Ex. 1) | C | C | C | C | C | C | C |
| | | 1.5 | 5 | 18 | 27 | 45 | 55 | 63 |
| | | (Ex. | (Ex. | (Ex. | (Ex. | (Ex. | (Ex. | (Ex. |
| | | 10) | 11) | 12) | 13) | 14) | 15) | 16) |

Band part
Unit: N

These results clarify that the elasticity of the body part has greater influence than the elasticity of the band part, and particularly indicate that the body part is preferably formed of a material requiring a force of 45 N or more to exhibit an elongation of 150%.

### (3) Evaluation of Storage Portion Area

Treatment devices having three types of areas, as shown in FIGS. 3 and 4, were prepared. The treatment device of Experimental Example 1 (FIG. 4(a)) had one rectangular storage portion with an area of 123.5 cm². The treatment device of Experimental Example 2 (FIG. 3) had two adjacent rectangular storage portions, each of which had an area of 61.75 cm². The treatment device of Experimental Example 3 (FIG. 4(b)) had four rectangular storage portions adjacent in a grid pattern, each of which had an area of 30.9 cm². Each of these treatment devices was used for three days by ten subjects suffering from wrist pain, and evaluated in terms of the degree of wrist pain alleviation on the following three scales: very satisfactory: A, satisfactory: B, and unsatisfactory: C. The results are as follows.

TABLE 5

| | Exp. Ex. 1 | Exp. Ex. 2 | Exp. Ex. 3 |
|---|---|---|---|
| Evaluation | A | A | C |

These results demonstrate the effectiveness of the treatment devices whose body parts had one or more storage portions each having an area of 60 cm² or more.

### Example 3

The treatment device for the knee shown in the third embodiment was prepared. First, the materials thereof are explained.

### (1) Material

The same materials as those used in Example 1 were used. Thus, the explanation thereof is omitted.

### (2) Evaluation of Elasticity

To evaluate the elasticity, sheet materials (100 mm×50 mm) were prepared for the body part and band part. Each sheet material was pulled in the longitudinal direction using a tensile testing machine (AGS-H, produced by Shimadzu Corp.), and the force required to elongate the sheet material 150% was measured. The above-described materials were combined to produce treatment devices of the form shown in FIG. 5. Each of the thus-prepared treatment devices was used for one day by five subjects suffering from knee pain, and evaluated in terms of the degree of knee pain alleviation on the following three scales: very satisfactory: A, satisfactory: B, and unsatisfactory: C. The results are as follows.

TABLE 6

| Body | 115 (Ex. 9) | A | A | A | A | A | A | A |
|---|---|---|---|---|---|---|---|---|
| part | 102 (Ex. 8) | A | A | A | A | A | A | A |
| | 85 (Ex. 7) | A | A | A | A | A | A | A |
| | 63 (Ex. 6) | A | A | A | A | A | A | A |
| | 55 (Ex. 5) | A | A | A | A | A | A | A |
| | 45 (Ex. 4) | B | B | B | B | B | B | B |
| | 27 (Ex. 3) | C | C | C | C | C | C | C |
| | 5 (Ex. 2) | C | C | C | C | C | C | C |
| | 1.5 (Ex. 1) | C | C | C | C | C | C | C |
| | | 1.5 | 5 | 18 | 27 | 45 | 55 | 63 |
| | | (Ex. | (Ex. | (Ex. | (Ex. | (Ex. | (Ex. | (Ex. |
| | | 10) | 11) | 12) | 13) | 14) | 15) | 16) |

Band part
Unit: N

These results clarify that the elasticity of the body part has greater influence than the elasticity of the band part, and particularly indicate that the body part is preferably formed of a material requiring a force of 45 N or more to exhibit an elongation of 150%.

### (3) Evaluation of Storage Portion Area

Treatment devices having three types of areas, as shown in the drawings, were prepared. The treatment device of Experimental Example 1 (FIG. 6(a)) had a rectangular storage portion in each storage area, each of which had an area of 123.5 cm². The treatment device of Experimental Example 2 (FIG. 5) had two adjacent rectangular storage portions in each storage area, each of which had an area of 61.75 cm². The treatment device of Experimental Example 3 (FIG. 6(b)) had four rectangular storage portions adjacent in a grid pattern in each storage area, each of which had an area of 30.9 cm². Each of these treatment devices was used for three days by ten subjects suffering from knee pain, and evaluated in terms of the degree of knee pain alleviation on the following three scales: very satisfactory: A, satisfactory: B, and unsatisfactory: C. The results are as follows.

TABLE 7

| | Exp. Ex. 1 | Exp. Ex. 2 | Exp. Ex. 3 |
|---|---|---|---|
| Evaluation | A | A | C |

These results demonstrate the effectiveness of the treatment devices whose body parts had one or more storage portions each having an area of 60 cm² or more.

### REFERENCE SIGNS LIST

1. Body part
11. Storage portion
2. Band part
3. Heating element
4. Surface fastener (fastening means)

1. A treatment device accommodating one or more heating elements that generate heat, and attachable to a treatment site of a patient, the device comprising:
a body part formed of an inelastic and breathable material, and having one or more storage portions individually storing each heating element;
one or more band parts formed of an elastic and breathable material, and attached to the body part; and
a fastening means for fastening the one or more band parts wound around the treatment site.

2. The treatment device according to claim 1, wherein the body part has one storage portion or two to four adjacent storage portions, per treatment site.

3. The treatment device according to claim 1, wherein the body part has one storage portion or two adjacent storage portions, per treatment site.

4. The treatment device according to claim 2, wherein the storage portions are arranged in a rectangular configuration in the body part, per treatment site.

5. The treatment device according to claim 2, wherein the one or more storage portions each have an area of 60 cm² or more.

6. The treatment device according to claim 1, wherein the body part has the one or more band parts at two opposite ends thereof.

7. The treatment device according to claim 1, wherein a pair of the band parts is attached to one end of the body part in parallel at a distance from each other.

8. The treatment device according to claim 3, wherein the storage portions are arranged in a rectangular configuration in the body part, per treatment site.

9. The treatment device according to claim 3, wherein the one or more storage portions each have an area of 60 cm² or more.

10. The treatment device according to claim 2, wherein the body part has the one or more band parts at two opposite ends thereof.

11. The treatment device according to claim 3, wherein the body part has the one or more band parts at two opposite ends thereof.

12. The treatment device according to claim 2, wherein a pair of the band parts is attached to one end of the body part in parallel at a distance from each other.

13. The treatment device according to claim 3, wherein a pair of the band parts is attached to one end of the body part in parallel at a distance from each other.
